SEQUENCE LISTING (16 sequences)

SEQ ID NO: 1 - DNA, 21 bp, Artificial Sequence
  Oligonucleotide primer
gtgacaggcaaggccgattcg

SEQ ID NO: 2 - DNA, 21 bp, Artificial Sequence
  Oligonucleotide primer
ttggacagtccagggctcagc

SEQ ID NO: 3 - DNA, 22 bp, Artificial Sequence
  Oligonucleotide primer
actcctggcacatgcctttgcc

SEQ ID NO: 4 - DNA, 22 bp, Artificial Sequence
  Oligonucleotide primer
taatcctcggtggtgccacacc

SEQ ID NO: 5 - DNA, 30 bp, Artificial Sequence
  Oligonucleotide primer
tttgctagtgtttaccacggatgccaacac

SEQ ID NO: 6 - DNA, 28 bp, Artificial Sequence
  Oligonucleotide primer
cctttgtagcggacgcaggagaagtcat

SEQ ID NO: 7 - DNA, 25 bp, Artificial Sequence
  Oligonucleotide primer
cgaatggctgtgaaggtgagattga

SEQ ID NO: 8 - DNA, 30 bp, Artificial Sequence
  Oligonucleotide primer
cagtggttccaggtatcagggctgtaaaat

SEQ ID NO: 9 - DNA, 30 bp, Artificial Sequence
  Oligonucleotide primer
caagccttcagtgagagccaagaaacaaac

SEQ ID NO: 10 - DNA, 32 bp, Artificial Sequence
  Oligonucleotide primer
cgtcatactcctgcttgctgatccacatctgc

SEQ ID NO: 11 - DNA, 30 bp, Artificial Sequence
  Oligonucleotide primer
caaacctgcagtcaatagccaacaggaaaa

SEQ ID NO: 12 - DNA, 32 bp, Artificial Sequence
  Oligonucleotide primer
atctggcaccacaccttctacaatgagctgcg

SEQ ID NO: 13 - DNA, 21 bp, Artificial Sequence
  Oligonucleotide primer
caaggcggcaatgaccactcc

SEQ ID NO: 14 - DNA, 21 bp, Artificial Sequence
  Oligonucleotide primer
ggcatcggcaaagtggtcaag

SEQ ID NO: 15 - DNA, 27 bp, Artificial Sequence
  Oligonucleotide primer
ggagaacagaattggttcctactttgg

SEQ ID NO: 16 - DNA, 30 bp, Artificial Sequence
  Oligonucleotide primer.  Y = C or T; W = A or T
cggagctccwatcacgaygtcattaaatcc

(19) United States
(12) Patent Application Publication
(10) Pub. No.: US 2004/0126825 A1
(43) Pub. Date: Jul. 1, 2004

(54) Methods of screening and using inhibitors of angiogenesis

(75) Inventors: Peter C. Baciu, Laguna Niguel, CA (US); Heying Zhang, Rockville, MD (US); Virna M. Manuel, Lawndale, CA (US)

Correspondence Address: Carlos A. Fisher, ALLERGAN, INC., T2-7H, 2525 Dupont Drive, Irvine, CA, 92612, US

(73) Assignee: Allergan, Inc

(21) Appl. No.: 10/697,487

(22) Filed: Oct. 29, 2003

Related U.S. Application Data
(63) Continuation of application No. 10/115,718, filed on Apr. 3, 2002.
(60) Provisional application No. 60/281,512, filed on Apr. 4, 2001.

(51) Int. Cl. G01N 33/574

(52) U.S. Cl. 435/7.23

(57) ABSTRACT

A method of screening for agents which are able to inhibit angiogenesis. Such agent have therapeutic application in the treatment of conditions including cancer, macular degeneration and retinopathies. Also included are methods of treating a patient having a pathological condition characterized by an increase in angiogenesis which comprises administering to the patient an agent capable of inhibiting activation of an integrin subunit.

This patent application is a continuation of co-pending application Ser. No. 10/115,718 filed on Apr. 3, 2002 which, in turn, claimed the benefit of U.S. Provisional Application Serial No. 60/281,512, filed Apr. 4, 2001, which is hereby incorporated by reference herein.

### BACKGROUND OF THE INVENTION

Angiogenesis is the method by which new blood vessels form from existing vasculature in an animal. The process is distinct from vasculogenesis, in that the new endothelial cells lining the vessel arise from proliferation of existing cells, rather than differentiating from stem cells. The process is invasive and dependent upon proteolyisis of the extracellular matrix (ECM), migration of new endothelial cells, and synthesis of new matrix components. Angiogenesis occurs during embryogenic development of the circulatory system; however, in adult humans, angiogenesis only occurs as a response to a pathological condition (except during the reproductive cycle in women).

Thus, in adults, angiogenesis is associated with conditions including wound healing, arthritis, tumor growth and metastasis, as well as in ocular conditions such as retinopathies, macular degeneration and corneal ulceration and trauma. In each case the progression of angiogenesis is similar: a stimulus results in the formation of a migrating column of endothelial cells. Proteolytic activity is focused at the advancing tip of this “vascular sprout”, which breaks down the ECM sufficiently to permit the column of cells to infiltrate and migrate. Behind the advancing front, the endothelial cells differentiate and begin to adhere to each other, thus forming a new basement membrane. The cells then cease proliferation and finally define a lumen for the new arteriole or capillary.

Due to the fact that certain pathologies including many cancers, retinopathies, arthritis, and macular degeneration depend upon angiogenesis, it would obviously be desirable to find methods for inhibiting angiogenesis associated with these conditions. Preferably such methods would not inhibit the angiogenesis involved in wound healing and other beneficial responses to angiogenic stimuli.

The matrix metalloproteases (MMPS) are a family of proteases that specifically degrade portions of the EMC. These secreted and membrane-associated extracellular proteins are widely considered to be involved in angiogenesis, probably being responsible, at least in part, for creating the opening in the ECM through which the growing vascular sprout can extend during angiogenesis. However, the specific molecular targets of the MMPs are the subject of some debate, as are the mechanisms by which the MMPs may influence other endothelial cell functions such as attachment to the ECM, detachment and migration.

Most MMPs are secreted as zymogens, which are activated in the ECM. The exception is MT1-MMP, which is bound to the cell surface and processed within the cell before migration to the cell membrane. A family of inhibitors of MMPs termed TIMPs (tissue inhibitors of metalloproteases) are antiangiogenic, but, having multiple and complex effects on the angiogenic process, they appear to possess activities in addition of those of a simple competitive inhibitor.

Formation of a vessel during angiogenesis requires the tight adhesion of neighboring endothelial cells in the basement membrane; this adhesion is mediated by members of the integrin superfamily. These transmembrane proteins consist of heterodimers comprising α and β subunits. There are various subtypes of each of the α and β subunits; thus a subunits may include α₃, α₄, α₅, α₆, α₇, α₈, α₉, α_{2b}, α_{E} and α_{V}, while the β subunits may include β₁, α₃, β₅, and β₆. As indicated in further detail below, there is specificity in most cases as to which α subtype can pair with which β subtype. Many, but not all, of the alpha subunits are expressed as an inactive pro form that is then cleaved by a protease termed convertase. Dimerization of these covertase-susceptible subunits appears to require convertase cleavage.

Endothelial cells express integrins in response to various factors including vascular endothelial growth factor (VEGF), transforming growth factor β (TGFβ) and basic fibroblast growth factor (bFGF). The expressed integrins mediate cell migration, proliferation, survival, and regulation of matrix degradation.

It has been reported that metalloprotease MT1-MMP, in conjunction with integrin αᵥβ₃, activates MMP-2 in cultured breast carcinoma cells by converting the latter from a pro-form to the active form of the enzyme. This activation is inhibited by the introduction of vitronectin, a specific ligand of αᵥβ₃. Deryugina E. I., et al., Exp Cell Res. 15;263(2):209-23 (February 2001). Additionally, it has been reported that MT1-MMP is capable of activating αᵥβ₃ by cleavage of the β₃ subunit when breast cells are transfected with MT1-MMP and the β₃ subunit. Deryugina E. I., et al., Int. J. Cancer 86(1):15-23 (April 2000). Both of these references are incorporated by reference herein.

### SUMMARY OF THE INVENTION

The present invention is related to the discovery that the matrix metalloprotease MT-1-MMP is capable of activating certain integrins by cleavage of the α subunit. We have discovered that this metalloprotease modifies the av subunit of integrin αᵥβ₃, the integrin widely thought to be associated with VEGF-mediated angiogenesis. Additionally, MT1-MMP is capable of activating, or increasing the activation state of, any α subunit that is susceptible to cleavage by convertase. Such subunits include α₃, α₄, α₅, α₆, α₇, α₈, α₉, α_{2b}, α_{E} and α_{V}. The MT1-MMP substrate may be the inactive pro-form of the α chain or may be the convertase-cleaved active form. In the latter case, MT1-MMP results in an increase in the activation state of the already active subunit.

Thus, MT1-MMP appears to be part of an angiogenic activation cascade involving integrin heterodimers. Such integrins may include, without limitation, α_{V}β₃, αᵥβ₁, α_{V}β₅, α_{V}β₆, and α₅β₁. As activation of integrin is a prerequisite for initiation of the angiogenic response, means of inhibiting such activation would be a valuable and useful therapeutic tool in the treatment of pathological conditions in which angiogenesis is at least partly a causative or perpetuating factor.

Thus, in one embodiment the invention relates to methods for screening agents which inhibit an angiogenic response comprising contacting together an inactive or convertase-activated integrin α subunit, an agent to be tested for the ability to inhibit angiogenesis, and metalloprotease MT1-MMP under conditions promoting the modification of the integrin α subunit in the absence of said agent, and correlating inhibition of an increase in α subunit activation with the ability of the agent to inhibit angiogenesis. In preferred embodiments, the MT1-MMP and pro form of the integrin α subunit are expressed within the same cell. Also, in a preferred embodiment, the correlating step is accomplished by observing a difference in migration of the MT1-MMP activated form versus the inactive form of the alpha subunit in electrophoresis or chromatography, as the former forms appear to migrate at a different molecular weight.

In another embodiment, the invention relates to a method of treating a patient suffering from a pathological condition in which angiogenesis is at least partially a causative or perpetuating factor with an agent capable of inhibiting an increase of a pro form or convertase-activated form of the integrin α subunit by MT1-MMP metalloprotease. In preferred embodiments, the pathological condition is selected from the group selected from arthritis, tumor growth, metastasis, retinopathies, macular degeneration, retinal neovascularization, corneal ulceration and corneal trauma.

In this embodiment of the invention, the agent may be administered by any means effective to direct the agent to the affected site. For example, without limitation, in the case of treatment of a tumor, the agent may be injected directly into tumor tissue, preferably into the periphery of the tumor mass; in the case or arthritis, the agent may be injected into the joint; in the case of ocular conditions the agent may be applied via an intraocular implant, such as a bioerodable or reservoir-based drug delivery system for direct treatment of the retina or cornea, or may be formulated in a ophthalmologically acceptable excipient and directly injected into the anterior or posterior segment of the eye.

### BRIEF DESCRIPTION OF THE DRAWINGS

FIG. 1 depicts a gel electrophoretogram of nucleic acid resulting from RT-PCR amplification of mRNA present in naiive corneas (lane 1), and 72 hours and 288 hours post cautery corneas (lanes 2 and 3 respectively. Oligonucleotide primers used corresponded to the labels in each row, and are shown in Table 1.

FIGS. 2A, 2C, 2E and 2G are photomicrograms of corneal tissue sections frozen 72 hours post-cauterization and immunostained with Factor VIII, fibronectin, laminin and tenacin-C, respectively.

FIG. 2B is a photomicrogram of a corneal tissue section frozen 72 hours post-cauterization and co-immunostained with Factor VIII and collagen type IV.

FIG. 2D is a photomicrogram of a corneal tissue section frozen 72 hours post-cauterization and immunostained with collagen type IV and fibronectin EDA.

FIG. 2F is a photomicrogram of a corneal tissue section frozen 72 hours post-cauterization and co-immunostained with collagen type IV and laminin.

FIG. 2H is a photomicrogram of a corneal tissue section frozen 72 hours post-cauterization and co-immunostained with collagen type IV and tenascin-C.

FIGS. 3A, 3C, 3E, and 3G are photomicrograms of tissue sections of the limbal region of naïve corneas immunostained for the α₁, α₂, α₅ and α₅ integrin subunits, respectively.

FIGS. 3B, 3D, 3F, and 3H are photomicrograms of central corneal region of naïve corneas immunostained for the α₁, α₂, α₅ and β₅ integrin subunits, respectively.

FIGS. 4A, 4E and 41 are photomicrograms of corneal tissue samples frozen 72 hours post-cautery and immunostained for α₁, α₂ and β₅ integrin subunits, respectively.

FIGS. 4C, 4G and 4K are photomicrograms of corneal tissue samples frozen 120 hours post-cautery and immunostained for α₁, α₂ and β₅ integrin subunits, respectively.

FIGS. 4B, 4F and 4J are photomicrograms of corneal tissue samples frozen 72 hours post-cautery and co-immunostained for a) collagen type IV, and b) α₁, α₂, and α₅ integrin subunits, respectively.

FIGS. 4D, 4H and 4L are photomicrograms of corneal tissue samples frozen 120 hours post-cautery and co-immunostained for a) collagen type IV, and b) α₁, α₂, and β₅ integrin subunits, respectively.

FIG. 5A is a photomicrogram of corneal tissue samples frozen 72 hours post-cautery and immunostained for the α₅ integrin subunit.

FIG. 5B is a photomicrogram of corneal tissue samples frozen 72 hours post-cautery and immunostained for collagen type IV and the α₅ integrin subunit.

FIG. 5C is a photomicrogram of corneal tissue samples frozen 120 hours post-cautery and immunostained for the α₅ integrin subunit.

FIG. 5D is a photomicrogram of corneal tissue samples frozen 120 hours post-cautery and immunostained for collagen type IV and the α₅ integrin subunit.

FIG. 5E is a photomicrogram of corneal tissue samples frozen 168 hours post-cautery and immunostained for the α₅ integrin subunit.

FIG. 5F is a photomicrogram of corneal tissue samples frozen 168 hours post-cautery and immunostained for collagen type IV and the α₅ integrin subunit.

FIG. 5G is a photomicrogram of corneal tissue samples frozen 72 hours post-cautery and immunostained for the integrin β₃ subunit.

FIG. 5H is a photomicrogram of corneal tissue samples frozen 72 hours post-cautery and immunostained for collagen type IV and the integrin β₃ subunit.

FIG. 5I is a photomicrogram of corneal tissue samples frozen 120 hours post-cautery and immunostained for the integrin β₃ subunit.

FIG. 5J is a photomicrogram of corneal tissue samples frozen 120 hours post-cautery and immunostained for collagen type IV and integrin B₃ subunit.

FIG. 6A is a confocal photomicrogram of whole mounted corneal tissue immunostained for lectin and integrin B₃ subunit in an alkaline burn model; wherein angiogenesis was induced by bFGF in the cornea.

FIG. 6B is a confocal photomicrogram of whole mounted corneal tissue samples immunostained for lectin and integrin B₃ subunit in an alkaline burn model, wherein angiogenesis was induced by bFGF in the cornea.

FIG. 6C is a confocal photomicrogram of whole mounted corneal tissue samples immunostained for lectin, wherein angiogenesis was induced by bFGF in the cornea. (L) is the limbus and (P) is the location of the pellet containing bFGF.

FIG. 6D is a confocal photomicrogram of whole mounted corneal tissue samples immunostained for integrin B₃ subunit, wherein angiogenesis was induced by bFGF in the cornea. (L) is the limbus and (P) is the location of the pellet containing bFGF.

FIG. 6E is a confocal photomicrogram of whole mounted corneal tissue samples immunostained for integrin B₃ subunit, wherein angiogenesis was induced by bFGF in the cornea.

FIG. 6F is a confocal photomicrogram of whole mounted corneal tissue samples immunostained for lectin and integrin B₃ subunit, wherein angiogenesis was induced by bFGF in the cornea.

FIG. 7A is a graphical representation of sections taken through naive and injured corneas.

FIG. 7B shows photographs of gelatin zymography from corneas taken from naïve corneas and corneas taken 24, 72, 120, and 168 hours post injury.

FIGS. 8A-E shows the results of in situ gelatin zymography in naive corneas and those injured 24 hours, 72 hours, 120 hours, and 168 hours post-injury, respectively.

FIGS. 9A-D are immunohistograms of frozen corneal sections frozen 72 hours post-injury. FIGS. 9A is stained form MMP-2 and FIG. 9C is stained for MT1-MMP. FIGS. 9B and 9D are stained for lectin, as well as MMP-2 and MT1-MMP, respectively.

The following examples do not limit the generality of the invention disclosed herein.

### EXAMPLES

Methods. Neovascularization in female sprague-dawley rats was induced by alkaline cauterization of the central cornea. Corneas from naïve, 72 hrs and 288 hrs post cautery animals were analyzed by RT-PCR for integrins α₁, β₂, β₃, β₅, the endothelial marker CD31, and metalloproteinases MMP-2 and MT1-MMP. Analysis of protein expression and metalloproteinases were conducted in corneas from naive, 24, 72, 120, and 168 hrs post cautery animals by immunofluorescent microscopy in frozen sections and gelatin zymography.

Results. RT-PCR indicated a correlation between expression of CD31, MT1-MMP and integrins α₁ and α₃, with neovascularization of the cornea. Immunohistochemical analysis indicated that at the protein level integrins α₁, α₂, α₅ and β₅, and MT1-MMP were expressed on newly developing vasculature while β₃ integrin was expressed at low levels within the neovascular lumen. As previously seen ECM proteins laminin, collagen type IV and fibronectin were expressed throughout the developing vasculature, however, tenascin-C showed preferential staining of maturing vasculature with little or no expression within the invasive angiogenic front. Expression of MMP-9 correlated with corneal epithelial cell migration while MMP-2 expression was associated with inflammatory cell invasion and neovessel formation.

Conclusions. Integrin expression during neovascularization of rat corneas in response to alkaline injury is restricted to angiogenesis along the VEGF/αᵥβ₅ pathway in conjunction with α₁β₁, α₂β₁ and α₅β₁ integrins. Expression of MT1-MMP within the invasive angiogenic front further suggest that MT1-MMP is also important in mediating VEGF driven angiogenic response, potentially in conjunction with αᵥβ₅ or β₁ integrins which co-distribute with MT1-MMP. The pattern of Integrin expression observed within this study correlates well with a VEGF mediated angiogenic response.

Angiogenesis within adult tissues is a response to a diverse set of stimuli including angiogenic and inflammatory cytokines that induce a quiescent vasculature to reenter the cell cycle and invade the surrounding stroma producing a new region of vascularized tissue. Central to this process are the activities of both cell adhesion receptors and matrix degrading enzymes belonging to the family of matrix metalloproteinases (MMPs). Inhibition or disruption of either cell adhesion or MMP activity through genetic manipulations or pharmaceutical intervention is capable of inhibiting an angiogenic response. In many instances the adhesion receptors involved and or MMPs are likely to be dictated by the angiogenic factors present. While this factor dependence has not been well characterized for MMPs, cell adhesion through integrins has been characterized to occur through at least two principle adhesion pathways corresponding to angiogenic induction by either bFGF or VEGF. Thus, in bFGF induced response, which also includes induction by TNF-α, angiogenesis occurs in an (vX3 mediated pathway, induction of angiogenesis by VEGF, as well as TGF-β and PMA, occurs through αᵥβ₅. While these two pathways are well established, recent studies suggest that under pathological conditions the correlation between growth factors and integrin expression are not always maintained. In several instances where VEGF is present both αᵥβ₃ and αᵥβ₅ are expressed and in at least one study the functional significance of αᵥβ₃ mediated angiogenesis may reflect the presence of ligand for (XvP3. Additionally, not all aspects of angiogenesis are dependent on expression of αᵥβ₃ or αᵥβ₅ integrins. Knockout mice for αᵥ as well as β₃ integrin appear to under go extensive vasculogenesis and angiogenesis in the absence of either αᵥ or αᵥβ₃ integrins, although subtle vascular defects are present with both embryonic and post natal lethality observed in association with abnormal vessel formation. These later observations suggest that other integrin family members are capable of complementing the functions of αᵥ or β₃ integrins or that other adhesive pathways, independent of αᵥ or β₃ integrins, are present. Other members of the integrin family implicated in mediating an angiogenic response include α₁β₁, α₂β₁, and α₅β₁ integrins which like αᵥ integrins have also been divided into bFGF associated (α₅β₁) or VEGF associated (α₁β₁, α₂β₁) angiogenic events. The above studies suggest that within a given angiogenic response the adhesion mediated pathway is likely to be diverse and depend not only on the presence of a single angiogenic factor but the collective influence of ECM and associated factors including MMPs and inflammatory cytokines.

Recently, the corneal alkaline burn model of angiogenesis has been characterized as having high levels of VEGF present during active vessel growth, suggesting that VEGF is the primary angiogenic factor within this model system. Consistent with this finding, pharmaceutical intervention with αᵥβ₃ antagonists has no effect on the angiogenic response, suggesting that angiogenesis occurs through an αᵥβ₅ adhesion pathway which is consistent with a VEGF mediated angiogenic response. However, expression of αᵥβ₅ was neither established in these studies nor other potential adhesion receptors identified. The purpose of this study was to characterize the pattern of integrin expression to determine if this angiogenic response occurs through a (v05 mediated pathway as well as characterize other members of the integrin family which may also be functionally relevant to a VEGF mediated angiogenic response. This study addresses these issues by examining both the spatial and temporal expression patterns of integrins relative to the expression of extracellular matrix molecules associated with a neovascular response including collagen type IV, laminin, fibronectin and tenascin-C. Additionally, we have examined the expression of metalloproteinases MMP-2 and MT1-MMP to determine if they are also involved in mediating the angiogenic response.

In conclusion, collagen type IV, laminin and fibronectin EDA domain expression was consistent with previous studies on neovascularization. Tenascin-C, however, showed a unique pattern of expression correlating with vessel maturation. In agreement with a VEGF mediated angiogenic response neovascularization was associated with expression of αᵥβ₅, α₁β₁ and α₂β₁ integrins as well as α₅β₁. MMP-2 and MT1-MMP were both associated with the robust inflammatory response as well as vessel formation. The localization of MT1-MMP to the developing vasculature in the absence of αᵥβ₃ suggests that MMP-2 as well as MT1-MMP may have broader roles in mediating an angiogeneic response than previously recognized by their association with αᵥβ₃ integrins.

Materials and Methods:

Reagents and antibodies: Brdu (5-bromo-2-deoxyuridine) was purchased from Boehringer Mannheim. TRIzol reagent and SuperScript II reverse transcriptase were from Gibco-BRL (Rockville, Md.). Gelatin zymography gels (10% PAGE), renaturing buffer and developing buffer were from Novex (San Diego). Primary antibodies were purchased from the following companies and used at the following concentrations: goat anti-type IV collagen was from Southern Biotechnology Associates, Inc. (Birmingham, Ala.) and used at 1:250 dilution (1.6 ug/ml); Mouse anti-fibronectin EDA domain, FN-3E2 was from sigma (St. Louis, Mo.) and used at 1:300 dilution, rabbit anti-human factor VIII was from Dako Corporation (Carpinteria, Calif.) and used at 1:100 dilution Anti-tenascin-C polyclonal antibody HXB1005 was a generous gift from: Sharifi B. G., and was used at 1:100 dilution; rabbit polyclonal anti-integrin α₁, subunit, -integrin α₂ subunit, -integrin α₃ subunit, -integrin α₅ subunit, -integrin 5 subunit were from Chemicon International Inc. (Temecula, Calif.) and used at 1:100 dilutions for the α₅ subunits and 1:500 dilution for 5 subunit; mouse monoclonal anti-rat integrin 3 chain was from PharMingen (San Diego, Calif.) and used at 1:100 dilution (5 ug/ml); rabbit polyclonal anti-MMP-2, and MT-MMP1 were from Chemicon International Inc. (Temecula, Calif.) All secondary antibodies were F(ab′)2 fragments conjugated to either rhodamine (TRITC) or fluorescein (FITC). They were purchased from Jackson ImmunoResearch Laboratories, Inc. (West Grove, Pa.) and used at 1:200 dilutions.

Animal model. Female rats (Sprague-Dawley), weighing 250-300 gm, were anesthetized with isoflurane (4% v/v) and topical application to the corneal surface with proparacaine 0.1% Allergan Inc. (Irvine, Calif.). The alkaline burn is created by touching the central cornea with the tip of a silver nitrate applicator (75% silver nitrate, 25% Potassium nitrate) Grafco™ Graham-Field Inc, (Hauppauge, N.Y.) for 2 seconds. At the indicated times animals were euthanized and the eyes were enucleated at post injury intervals ranging from 24 hrs to 288 hrs for various studies. For immunofluorescence analysis, the eyes were embedded in OCT solution and cryosectioned. For wholemount studies, entire corneas were removed and quartered. Experimental animals were treated and maintained in accordance with ARVO statement for the Use of Animals in Ophthalmic and Vision Research.

Cryosectioning and Immunofluorescence. The eyes (injured or naïve) were sagittally cryosectioned in 8-13 μm sections for immunostaining with mouse monoclonal or goat and rabbit polyclonal antibodies. The sections were fixed in 100% acetone for 5 minutes, briefly dried, rehydrated in phosphate-buffered saline (PBS) and incubated in a moist chamber as follows: 5% BSA (Sigma) in PBS for 2 hr, primary antibodies for 2 hr at room temperature, five washes in PBS for 5 min each, secondary antibodies conjugated to fluorochromes for 1 hr at room temperature, five washes as before. Samples were mounted with Fluoromount G (Southern Biotechnology Associates) and observed and photographed with a Nikon E800 compound microscope equipped with a Spot Digital Camera (Diagnostic Instruments Inc. Sterling Heights, Mich.). Co-localization of the angiogenesis-related molecules and vascular markers were achieved by using various combinations of mouse, goat or rabbit primary antibodies. Negative controls for immunostaining were the use of naive serum or purified IgG for each species of primary used as well as secondary alone. In all instances tissues were co-stained with Collagen type IV to mark the presence of vessels as well as serve as an internal positive control. All control tissues were from corneas 72 hrs post injury since this provided the greatest range of cellularity.

Whole Mount Immunofluorescence: Complete fresh corneas were cut in quarters and fixed in 90% methanol and 10% DMSO for 15 min at room temperature, rinsed in PBS (1×) 2 min×3 times, blocked in 2% BSA in PBS for 4 hrs, incubated in primary antibody α2/CD31 or β5/CD31, β3/Banderaea Simplicifolia (BS-1) lectin overnight at 4° C., washed in PBS 1 hr×5 times, followed by incubation in second antibodies conjugated to fluorochromes for overnight at 4° C. and washed for 1 hr×5 times. Finally corneas were flat mounted and analyzed by either a Nikon E800 compound microscope equipped with a Spot Digital Camera (Diagnostic Instruments Inc. Sterling Heights, Mich.) or by Confocal microscopy using a Lecia TCS SP confocal microscope (Leica Microsystems Inc., Exton, Pa.).

In Situ Zymography: Frozen tissue sections, 4-8 um in thickness were mounted onto gelatin coated slides (Fuji, Pharmaceuticals Inc.) and incubated at 37° C. in a moist chamber for 4 hrs to 6 hrs followed by drying at room temperature. After fixation, tissues were stained with Amido Black 10B solution for 15 minutes followed by rinsing in water and then destain (70% methanol, 10% acetic acid) for 20 minutes. Images were captured by bright field microscopy.

RT-PCR: The total RNA was isolated from the pooled corneal tissue (total of four corneas) from naive, 72 hr and 288 hrs post cautery animals using a standard TRIzol extraction procedure as outlined in the manufacturer's protocol GibcoBRL (Rockville, Md.). Isolated RNA was treated with Rnase free DNase I to remove any contaminating genomic DNA. RT-PCR analysis of RNA in the absence of reverse transcriptase was used as a negative control. The total RNA was quantitated by spectrophotometry at an absorbence of 260 nm. Total RNA (1 μg) was reverse transcribed with 50 units SuperScript II reverse transcriptase in the presence of 2.5 ug/ml random hexamer and 500 μM DNTP for 50 min at 42° C., followed at 70° C. for 15 min. 1 ul of the resulting cDNA was amplified in the presence of 1 nM sense and antisense primers, 200 uM DNTP, and 3.5 units of Expand™ High Fidelity enzyme mix —PCR conditions: Initial 5 cycles, denature at 94° C. for 15 sec, annealing at 58-55° C. for 30 sec (decrease 0.5° C. each cycle), and 72° C. for 30 seconds. For the remaining 27 cycles PCR conditions were 94° C. for 15 sec, 55° C. for 30 sec, and 72° C. for 45 seconds. The amplified samples were then loaded at equal volumes (10 μl) onto 1.5% agarose gels. The PCR products were visualized with ethidium bromide. The primer pairs used for amplification are given in Table 1. All PCR products were subcloned and sequenced to verify product as the target gene.

Corneal Micropocket Assay: Corneal Micropocket assay was carried out as described in (23) using 400 ng bFGF/hydron pellet bead. Briefly, Female rats (Sprague-Dawley), weighing 250-300 gm, were put under general anesthetized with 200 μl of (xylazine 20 mg/ml, Ketamine 100 mg/ml and acepromazine) and prior to surgery eyes were topically anesthetized with 0.5% proparacaine. A 1 mm in length corneal incision penetrating half through corneal stroma was made 2.5 mm from the temporal limbus and a pocket was made by separating stroma from the point of incision to about 1 mm from limbal vessel. A hydron bead 0.4×0.4 mm containing 140 ng bFGF was then implanted in the pocket. Three and five days after implantation of hydron pellet corneas were prepared for whole mount analysis.

TABLE 1

Oligonucleotide Primer Sequences

| | | |
|---|---|---|
| | | Fragment size |
| Primer | Oligonucleotide Sequence | (bp) |

| | | | |
|---|---|---|---|
| MT1-MMP: | 5′-GTGACAGGCAAGGCCGATTCG-3′ | SEQ. ID NO. 1 | 446 |
| | 5′TTGGACAGTCCAGGGCTCAGC-3′ | SEQ. ID NO. 2 | |
| MMP-2, | 5′-ACTCCTGGCACATGCCTTTGCC-3′ | SEQ ID NO. 3 | 401 |
| | :5′-TAATCCTCGGTGGTGCCACACC-3′ | SEQ. ID NO. 4 | |
| integrin β3 | 5′-TTTGCTAGTGTTTACCACGGATGCCAACAC-3′ | SEQ. ID NO. 5 | 866 |
| | 5′-CCTTTGTAGCGGACGCAGGAGAAGTCAT-3′ | SEQ ID NO. 6 | |
| integrin β5 | 5′-CGAATGGCTGTGAA GGTGAGATTGA-3′ | SEQ ID NO. 7 | 854 |
| | 5′-CAGTGGTTCCAGGTATCAGGGCTGTAAAAT-3′; | SEQ ID NO. 8 | |
| integrin α2 | 5′-CAAGCCTTCAGTGAGAGCCAAGAAACAAAC-3′ | SEQ ID NO. 9 | 728 |
| | 5′-CAAACC TGCAGTCAATAGCCAACAGGAAAA-3′ | SEQ ID NO. 10 | |
| integrin α1 | 5′-GGAGAACAGAATTGGTTCCTACT TTGG-3′ | SEQ ID NO. 11 | 335 |
| | 5′-CGGAGCTCCWATCACGAYGTCATTAAATCC-3′ | SEQ ID NO. 12 | |
| CD31 | 5′-GGCATCGGCAAAGTGGTCAAG-3′ | SEQ ID NO. 13 | 680 |
| | CAAGGCGGCAATGACCACTCC | SEQ ID NO. 14 | |
| Actin | 5′-ATCTGGCACCACACCTTCTACAATGAGCTGCG-3′ | SEQ ID NO. 15 | 837 |
| | 5′-CGTCATACTCCTGC TTGCTGATCCACATCTGC-3′ | SEQ ID NO. 16 | |

W = A or T, Y = C or T.

Results

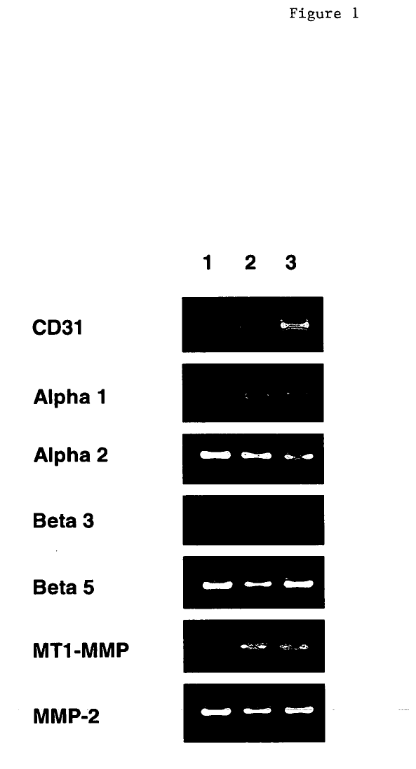

To examine the presence or absence of individual integrins and MMPs, RT-PCR was performed examining integrins α₁, α₂, β₃, β₅ and metalloproteinases MMP-2 and MT1-MMP using naive, 72 hrs (3 days) and 288 hrs (12 day) post cautery corneas. This allowed examination of tissues representing the early (72 hrs) and late phases (288 hrs) of the angiogenic response. Correlation between gene expression relative to vessel growth was accomplished by examining the expression of CD31. Analysis of naive cornea indicated the absence of messages for CD31, α₁, β₃, and MT1-MMP. Message for MMP-2, β₅, and α₂ integrin was present in naive corneas (FIG. 1). Within injured cornea at both early and late phases of neovascularization α₁, β₃, MT1-MMP, and CD31 mRNA were detected. The correlation between α₁,β₃, MT1-MMP with CD31 expression suggests involvement of the encoded proteins with the neovascular response. Expression of MMP-2, β₅ integrin, and α₂ integrin messages showed no clear change in expression with neovascularization. The absence of a correlation between MMP-2, β₅, and α₂ mRNA with the angiogenic response does not exclude their potential involvement within the angiogenic response but is likely to reflect the limitation of the approach and the relatively high levels already present in naïve corneas. To further refine the analysis, protein expression was examined by immunohistochemical analysis in conjunction with gelatinase zymography. To map the expression of integrins and MMPs to the developing vasculature corneal tissue sections were initially stained for factor VII to identify endothelial cells as well as a number of extracellular matrix proteins associated with a neovascular response including collagen type IV, laminin, fibronectin EDA domain and tenascin-C.

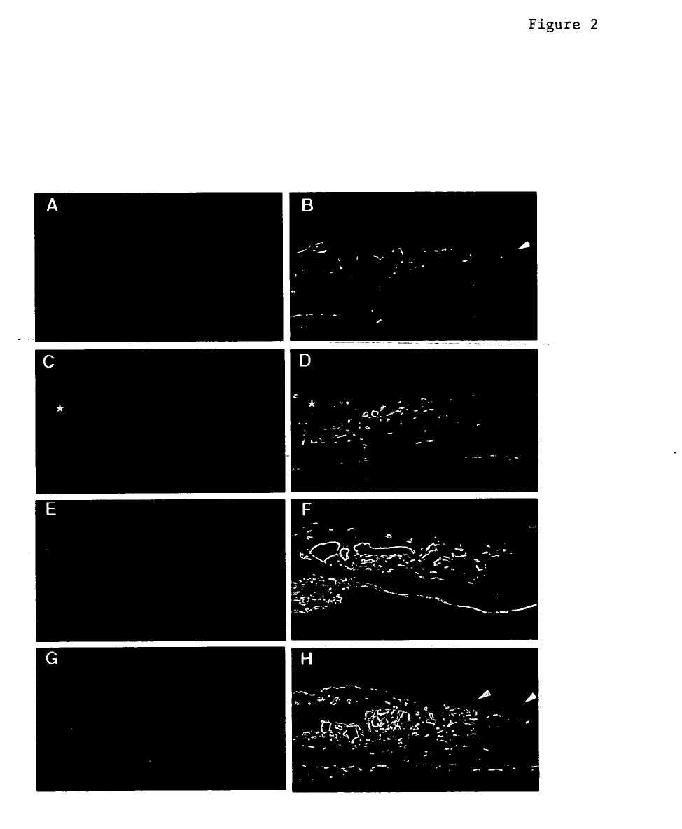

Staining in frozen tissue sections from corneas 72 hrs post injury with Factor VIII, collagen type IV, fibronectin EDA domain, laminin and tenascin-C are presented in FIG. 2. The entire vasculature as well as distal regions of the developing vasculature were positive for factor VII, co-immunostaining with collagen type IV showed a similar pattern of vessel staining as that seen with factor VIII, however, collagen type IV did not stain the more distal regions recognized by factor VIII immunostaining (FIG. 2B, arrow head), indicating the invasive front proceeds pronounced collagen type IV expression but is factor VIII positive. Coinciding with collagen type IV staining was staining for fibronectin EDA and laminin (FIGS. 2C-2F). The one exception to the staining observed between collagen type IV, laminin and fibronectin EDA domain was the absence of fibronectin EDA domain staining in the limbal or pre-existing vasculature (FIGS. 2C and 2D, asterisk). Tenascin-C expression (FIGS. 2G and 2H), while present within the limbal vasculature, was initially expressed proximal to the initial expression seen for collagen type IV in which a region of collagen type IV positive and tenascin-C negative could be recognized in the more distal regions of vessel formation (FIG. 2H, between arrow heads). The rather high levels of tenascin-C seen in the stroma represent remnants of tenascin-C from the scaleral spurr which is rapidly degraded during the initial 24 hrs after corneal cauterization. This later response is restricted to the cautery burn injury as a simple corneal debriment had no effect on the degradation of tenascin within the scaleral spur (data not shown). The staining pattern of ECM is consistent with that which as been previously reported for collagen type IV, fibronectin EDA and laminin, however, the localization of tenascin-C to more proximal regions of the developing vasculature has not been previously reported. The unique staining pattern of tenascin-C relative to collagen type IV allows identification of a unique region, which may represent a pre-maturation phase in vessel development. Based on the pattern and relative fluorescence intensity, collagen type IV was used to mark the developing vasculature in the following studies examining both integrin and MMP expression.

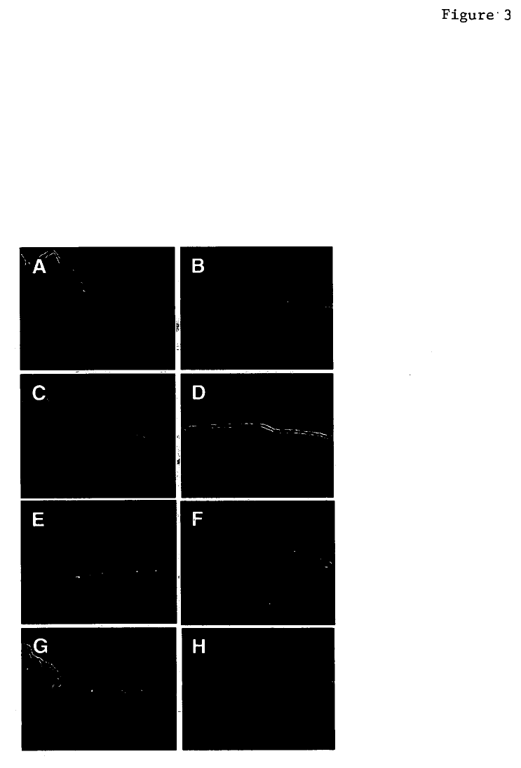

For the analysis of integrin expression immunological reagents were selected to identify a given integrin pairing. The heterodimer pairs examined in the current study are α₁α₃, α₂β₁, α₅β₁, αᵥβ₃, and αᵥβ₅. Identification of the respective heterodimers was accomplished by staining tissues for α₁, α₂, α₅, β₃, and β₅ integrin subunits. In most cases this allowed the identification of a discrete heterodimer pair since α₁, α₂, and α₅ only pair with β₁ integrin subunit and 135 only pairs with αᵥ subunit. The only exception being the anti-β₃ antibody which recognizes both the αᵥβ₃3 and α_{viib}β₃ heterodimer pairs. However, α_{iib}β₃ is only expressed on platelets and megakaryocytes allowing elimination based on cell morphology and tissue distribution. Corneas were examined from three separate time courses for each integrin in which cornea staining was examined in naive, 24, 72, 120 and 168 hrs post cautery. Shown for each of the staining patterns are the 72 and 120 hr time points as these represent the spectrum of staining observed throughout the time course and are believed to represent both early and mid phases of the angiogenic response. Staining in naive corneas for each of the integrins examined is shown in FIG. 3. The majority of staining was seen for α₁, α₂, α₅ and β₅ within the corneal epithelium. Stromal staining was also observed but to a limited extent and not readily apparent (FIG. 3). No immunoreactivity was seen for β₃ integrin (not shown).

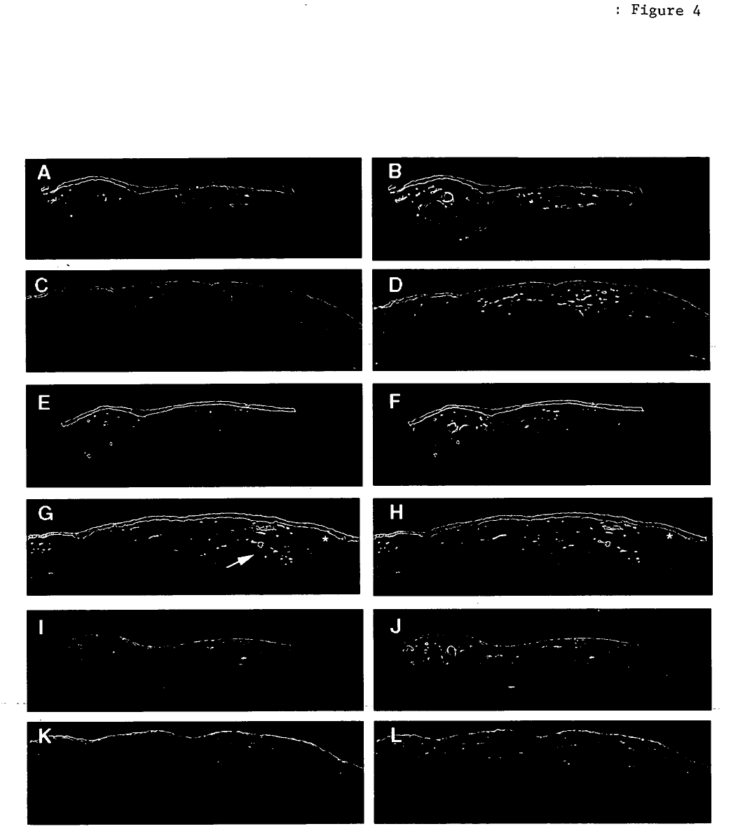

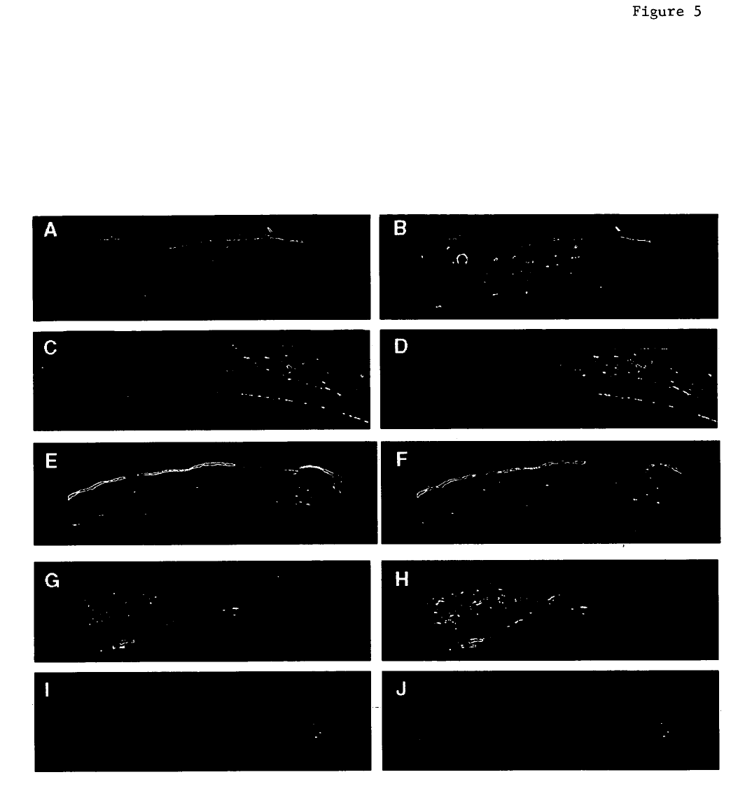

Staining patterns for α₁, α₂ and β₅ are shown for both the 72 hrs. and 120 hrs in the time points in FIG. 4. Examination of α₁, α₂, and β₅ at 72 hrs. post injury indicated similar patterns of expression with staining in the limbal vessels and throughout the developing vasculature co-localizing with collagen type IV immunostaining. Staining of cells within the stroma for α₁, α₂, and β₅ not directly associated with the neovessels was also observed (FIG. 4). This latter staining pattern is likely to represent the expression on stromal fibroblast or inflammatory cells which are highly abundant within the stroma at this time point. Expression of α₁ within the developing vasculature showed a uniform pattern of staining throughout the developing vasculature while that for α₂ was variable and punctate. At the 120 hrs. time point α₂ showed diminished staining within the leading vascular front (FIGS. 4G and 4H, asterisk) with pronounced staining within the vasculature frequently observed (FIG. 4G, arrow). This latter staining may reflect α₂ expression on platelets or inflammatory cells present within the neovessels. β₅ integrin staining in the developing vasculature was similar to α₁, with expression throughout the developing vasculature (FIGS. 4I-4L). At the 120 hrs. β₅ continued to show staining throughout the developing vasculature (FIGS. 4K and 4L). However, preferential staining in more distal regions of the developing vasculature was frequently observed.

Staining for α₅ integrin subunits identifies the presence of the α₅β₁ heterodimer since α₅ is only known to pair with the β₁ integrin subunit. This integrin heterodimer pair is expressed in multiple cell types and consistent with this pattern of expression α₁ is observed in corneal epithelial and endothelial as well as stromal cells in naïve and injured cornea. Similar to α₁, α₅ staining was uniform throughout the developing vasculature at the 72 hrs. time point (FIGS. 5A and 5B). At the 120 hrs. time point, α₅ showed localized staining in the more distal regions of the neovasculature (FIGS. 5C and 5D) and by 168 hrs this differential staining pattern was more pronounced (FIGS. 5E and 5F). These results from the α₁, α₂, α₅ and β₅ staining suggest within the more distal regions involved in vessel outgrowth, adhesion occurs through α₁β₁, α₅β₁ and αᵥβ₅ integrins. The Pattern of α₂ staining suggests its potential involvement in the early phases of the angiogenic response but by 120 hrs it is preferentially expressed in regions associated with vessel maturation and remodeling.

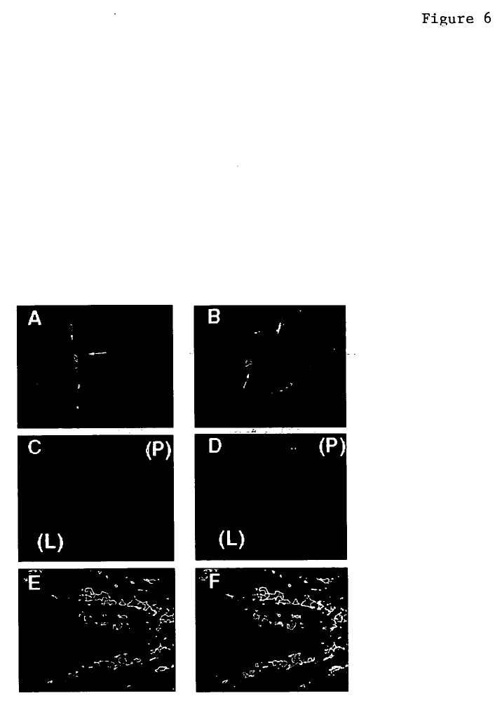

Staining for β₃ integrin subunits identifies the presence of either the αᵥβ₃ or 60 _{iib}β₃ heterodimers. Within naïve cornea β₃ immunostaining is absent (not shown). At 72 and 120 hrs. post injury faint β₃ staining was observed throughout the developing vasculature punctuated by regions of pronounced β₃ immunofluorescence (FIGS. 5G-5J). Confocal microscopy of whole mounted corneal tissues indicates that the pronounced β₃ immunostaining is associated with expression of β₃ on platelets (FIGS. 6A and 6B). To confirm that the staining pattern for β₃ is not associated with neovascularization we examined the expression of β₃ in which corneal angiogenesis was induced by bFGF using the corneal micropocket assay. Examination of the bFGF induced neovascularization indicates that β₃ expression is restricted to the leading vasculature front (FIGS. 6C and 6D) as well as pronounced expression on endothelial cells (FIGS. 6E and 6F). These results are consistent with previous studies examining (αᵥβ₃ expression in neovascularized tissue and contrasts greatly with the observed β₃ staining seen in the corneal alkaline burn model. These data indicate that β₃ is not expressed in a fashion consistent with its involvement in mediating endothelial cell adhesion to the extracellular matrix and that the observed β₃ expression is principally expressed on platelets as α_{iib}β₃.

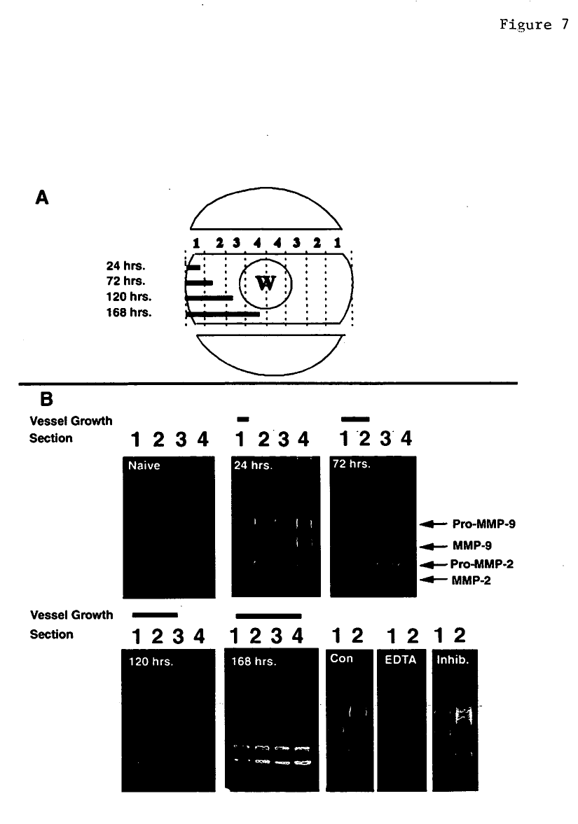

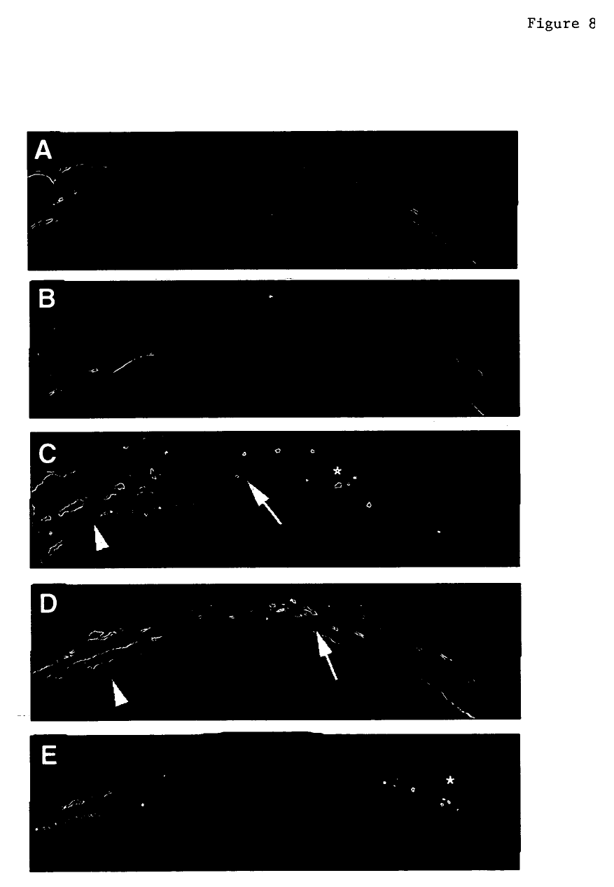

In addition to the expression of integrins identified by the RT-PCR analysis, message for MT1-MMP was also detected and the presence of this message correlated with neovascularization of the cornea. Since MT1-MMP is tightly associated with activation of MMP-2^{18,19} we initially examined potential involvement of MT1-MMP by examining the presence of the pro and activated forms of MMP-2 by gelatin zymography. Gelatin zymography was performed on corneas from naïve, 24 hrs, 72 hrs, 120 hrs and 168 hrs post injury. To correlate MMP expression with vessel growth corneas were sectioned as shown in FIG. 7A. This provided a relative reference of MMP activity to new vessel growth. In naive corneas only the pro-form of MMP-2 was present (FIG. 7B). At 24 hrs post injury, active forms of MMP-2 were detected in all sections with highest levels present within limbal and wound domains (FIG. 7B, sections 1 and 4). At 72 hrs. active forms of MMP-2 were more prevalent in the limbal and adjacent domains forming a gradient with highest levels in the limbal regions (FIG. 7B, Sections 1 and 2), suggesting a correlation between the presence of active forms of MMP-2 and neovessel formation. At 120 hrs. the gradient of active forms of MMP-2 extended into the central cornea and by 168 hrs. the gradient had reversed with highest levels seen in the central cornea (FIG. 7B, section 4). These data suggest a correlation between vessel growth and MMP-2 activation implicating an active role of MT1-MMP in the angiogenic process. In addition to MMP-2, MMP-9 expression and activity were also observed by gelatinase zymography. Within 24 hrs post injury pro and active forms of MMP-9 were detected though out the cornea with higher levels seen in sections 3 and 4, representing the wound and adjacent tissue. By 72 and 120 hrs. MMP-9 levels were greatly decreased with only the pro-form detected within the regions of the original corneal wound. This pattern of MMP-9 expression is consistent with expression of MMP-9 during corneal epithelial cell migration.

The complex pattern of MMP-2 activation observed is likely to reflect both active enzyme and that associated with TIMPS as an inactive complex. Additionally, MMP-2 activity is also like to be associated with inflammatory or stromal fibroblasts not directly associated with the angiogenic process. To identify endogenously active MMP-2 within the cornea in situ zymography was performed (FIG. 8). Consistent with the gelatinase zymography the pattern of gelatinase activity as determined by in situ zymography were very similar. In naive tissue no gelatinase activity was observed and by 24 hrs. a small increase in gelatinase activity was seen through out the cornea. At 72 hrs. gelatinase activity was present within the limbal (FIG. 8C, arrowhead) and adjacent regions (FIG. 8C, arrow) reflecting the gradient of active forms of MMP-2 observed in the gelatinase zymography. The extent of gelatinase activity extending into the corneal stroma correlates with neovessel formation as previously determined by collagen type IV immunostaining. Additionally, pronounced gelatinase activity was observed within individual cells within the stroma (FIG. 8C, asterisk). At 120 hrs. gelatinase activity was similar to that observed at 72 hrs. with the regions of stromal associated gelatinase activity extending further into the corneal stroma correlating with vessel development (FIG. 8D). At 168 hrs post injury the majority of gelatinase activity was restricted to individual cells within the central cornea adjacent to the wound. The relatively low levels of gelatinase activity between the limbus and central wound observed in the in situ zymography at 168 hrs. relative to the levels of active forms of MMP-2 observed by gelatin zymography (FIG. 7, 168 hrs. time point) suggests that gelatinase activity between the limbus and central cornea are tightly regulated by endogenous TIMPS, consistent with down regulation of MMP activity within regions of vessel maturation.²² Finally, in the in situ zymography little or no gelatinase activity was seen in relationship to the cornea epithelial cells, this may reflect the inability to obtain adequate development time to allow visualization of an MMP-9 signal. Longer development times often resulted in loss of resolution in the gelatinase activity.

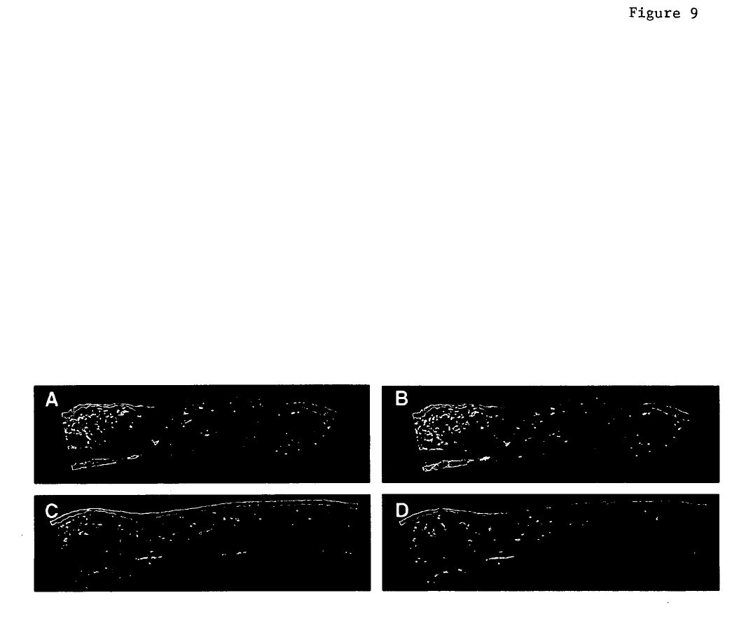

To further define the localization of MMP-2 and expression of MT1-MMP immunohistochemical staining was preformed on frozen corneal sections. Analysis indicated pronounced MPP-2 expression in individual cells within the stroma similar to that seen by in situ zymography with low levels of staining seen in association with developing vasculature (FIGS. 9A and 9B). MT1-MMP expression was similar to that seen with MMP-2 although higher levels were observed in association with the developing vasculature (FIGS. 9C and 9D). The strong staining of individual cells within the stroma for MMP-2 suggests that the gelatinase activity seen in the in situ zymography reflects active MMP-2. The gelatinase activity in association with vessel growth may reflect gelatinase activity associated with MMP-2 as well as M1-MMP, which showed pronounced staining on the developing vasculature correlating with in situ zymography.

Discussion

We examined the pattern of integrin and MMP expression within the corneal alkaline burn model relative to the angiogenic response by RT-PCR, immunofluorescence and gelatin zymography. Initial analysis of integrin and metalloproteinase expression by RT-PCR demonstrated that CD31, integrins α₁ and β₃, and MT1-MMP were expressed in injured cornea correlating with the angiogenic response seen within this model. Expression of α₂, β₅ and MMP-2 indicated no alteration in their pattern of expression relative to neovascularization. The inability to detect a change in message for α₂ integrin, β₅ integrin, and P-2 likely reflects the existence of abundant message present in naïve tissues. The expression of MMP-2, β₅ and α₂ in naive tissue likely reflects the expression of these genes within the corneal epithelium for β₅ and α₂ integrins or within the corneal stroma for MMP-2.

Having identified potential adhesion and metalloproteinases associated with the angiogenic response by RT-PCR we next examined their expression in relationship to vessel formation by immunohistochemical analysis. This was accomplished by initially examining vessel development using the endothelial cell marker factor VIII as well as a number of ECM proteins associated with neovessel development, this included collagen type IV, fibronectin EDA domain, tenascin-C and laminin. From this analysis collagen type IV, fibronectin EDA domain, and laminin stained the entire developing neovasculature with the exception of the more distal regions which were only positively stained for factor VIII. The absence of a clear basement membrane staining at the more distal regions of the developing neovessels is consistent with the observations of Paku and Paweletz, 1991 in which a defined basement membrane is absent within the invasive tips of vascular buds. The Pattern of collagen type IV, laminin and fibronectin expression is similar to that reported by others examining basement membrane formation during angiogenesis in adult tissue, although, we did not see preferential expression of laminin preceding collagen type IV as reported by Form et al., 1986 during alkaline burn induced corneal neovascularization in the mouse. Both collagen type IV and laminin as well as factor Vm stained the preexisting limbal vasculature while no staining for fibronectin EDA domain was seen. This is consistent with embryonic forms of fibronectin only being expressed in newly developing vasculature in adult tissues or within large vessels. Proximal to the initial staining by collagen type IV was staining of tenascin-C which extend throughout the developing vasculature and into the pre-existing limbal vasculature. This pattern of tenascin-C staining identifies a subdomain in the ontogeny of vessel development between the more distal regions as identified by factor VIII staining and more proximal regions which are positive for tenascin-C but negative for collagen type IV, Laminin and fibronectin EDA domain. This subdomain may represent a prematuration phase prior to the formation of a more stable vasculature marked by pronounced tenascin-C staining. Potentially, tenascin-C may support stable association of smooth muscle cells or pericytes with the developing vasculature, however, in several reports tenascin-C expression has been associated with endothelial sprouting and activation suggesting that tenascin-C may also be modulating active remodeling of the primitive capillary bed as well as stabilization of pericyte association.

Using collagen type IV as a marker for vessel formation the pattern of integrin expression was examined. Data analysis from frozen sections indicated that 3 integrin was principally expressed on platelets within the developing vasculature. The staining on platelets and not endothelial cells was confirmed by comparing β₃ staining from the corneal burn model with 3 staining induced by bFGF in the corneal micropocket assay. Based on these analysis the αᵥβ₃ integrin does not appear to play a functional role in endothelial cell mediated migration and angiogenesis within this model system. Further support for this conclusion is the recent report by Klotz et al., in which LM609, an αᵥβ₃ specific inhibitory antibody, failed to inhibit angiogenesis within this model system, although a modest but statistically significant inhibition was seen by LM609 in bFGF induced angiogenesis in the rat cornea. The presence of a β₃ specific band in the RT-PCR analysis may represent the expression of αᵥβ₃ on macrophages which are present in high abundance throughout the time period studied. Alternatively, the β₃ mRNA message detected by RT-PCR maybe the result of expression in endothelial cells, which showed a low level of staining localized to the lumenal surface. This may reflect a response of endothelial cells within this model similar to that observed in response to ischemic insult in which high levels of VEGF are also present. Functionally this may facilitate platelet or leukocyte adhesion within the developing neovasculature.

Within the developing neovasculature α₁, α₂ and α₅ integrins expression was seen to co-localize with collagen type IV in association with vessel formation at 72 hrs. At later time points (120-168 hrs) α₁ integrin was uniformly expressed within the developing neovasculature, while α₂ appeared to be more prevalent in regions of vessel maturation. The as integrin showed a preferential localization to the more distal regions of vessel formation suggesting a role for α₅β₁ integrin in the invasive and early maturation and remodeling phases of vessel development within this model system. The role of α₁ and α₂ during vessel formation and maturation maybe associated with regulation of MMP activity and increase in collagen synthesis as a new basement membrane is formed. Both α₁ and α₂ have also been shown to be essential for VEGF mediated angiogenesis and suggested to be expressed early in the angiogenic in response to VEGF. This also appears to be the case within this model system, however, in later phases of the angiogenic response only α₁ was consistently detected in the more distal regions of vessel formation associated with bud formation and endothelial cell invasion.

β₅ integrin staining was seen throughout the developing vasculature during the early and late phases of vessel formation, however, β₅ integrin appeared more prevalent within distal regions of the developing vasculature. These data suggest that in this model system the αᵥα₃ integrin is associated with vessel development and not αᵥβ₃. The association of α₁, α₂, and β₅ integrins in the angiogenic response in the corneal alkaline burn is in keeping with VEGF mediated angiogenic events¹² and the previously observed up regulation of VEGF expression associated with corneal angiogenesis. However, the presence of α₅ integrin within the nascent vasculature also suggests that α₅β₁ may also play a significant role, potentially in mediating endothelial cell invasion and tubule formation. Involvement of α₅β₁ in both endothelial cell migration and tubule formation has been demonstrated in in vitro model systems. Although, functional analysis in a VEGF driven pathway has failed to demonstrate an essential role for α₅β₁.

The other aspect of angiogenesis studied was the expression and activation of MMPs. Within this study the activities of three MMPs were examined. This included MMP-9, MMP-2 and MT1-MMP. Activities of MMP-9 and MMP-2 were addressed by gelatinase zymography and in situ zymography while that of MT1-MMP was inferred by the presence of active MMP-2 and positive immunostaining for MT1-MMP. Both MMP-2 and MT1-MMP were found to be present within this model system and based upon both zymographic and immunohistochemical analysis shown to be associated with the angiogenic response. The correlation between MMP-2 activation and MT1-MMP immunoreactivity suggests that MT1-MMP is associated with the activation of MMP-2 in this model system. While the data suggest that MT1-MMP is involved in MMP-2 activation other mechanisms of MMP-2 may also be present. Currently, MMP-2 and MT1-MMP are believed to form a functional complex in conjunction with αᵥβ₃ and TIMP-2 on the cell surface which in turn mediates localized pericellular proteolysis of the ECM facilitating direction migration and invasion of endothelial cells. Inhibition of this complex formation has been shown to inhibit an angiogenic response further establishing the functional importance of MT1-MMP and MMP-2 in mediating an angiogenic response. However, in the alkaline induced corneal angiogenesis model αᵥβ₃ does not appear to play a major role in mediating the angiogenic response and thus the role of MT1-MMP and MMP-2 within this models may function outside of their association with αᵥβ₃. Recently, MT1-MMP has been shown to directly mediate cell migration and adhesion through modulation of integrin activity independent of MMP-2. Potentially within the current model system, where αᵥβ₃ is not present, MT1-MMP may be directly regulating endothelial cell activity by modulating either αᵥβ₅ or beta 1 integrins that co-distribute with MT1-MMP in neovessels.

In addition to MMP-2 and MT1-MMP we also observed increased levels of MMP-9 for both the pro and activated forms. Both the temporal and spatial pattern of MMP-9 expression and activity suggested its association with wound healing and migration of corneal epithelial cells. This, however, does not eliminate a potential role of MMP-9 in regulating the angiogenic response through the generation of angiostatins or release of pro-angiogenic factors from the matrix. Whether MMP-9 plays either a pro-angiogenic or anti-angiogenic role in this model system remains to be determined. Potential activities associated with release of pro angiogenic factors maybe associated with the early degradation of tenascin-C in the scaleral spur which is observed within the initial 24 hrs after wounding. This response appears to be specific to the angiogenic response since simple corneal debriment does not result in degradation of tenascin-C within the scaleral spur.

In conclusion, the αᵥβ₅ integrin appears to be the principal αᵥ integrin associated with endothelial cells within the corneal alkaline burn model of inflammatory mediated angiogenesis. In addition to αᵥβ₅, the α₁β₁, α₂β₁, and α₅β₁ integrin showed consistent localization to the developing vasculature bed. Of particular significance was the preferential localization of α₅β₁ to more distal regions of the developing vasculature. Examination of tenascin-C staining suggests that tenascin-C expression is associated with vessel maturation and has allowed the identification of a novel domain between the invasive front and putative vessel maturation which is tenascin-c negative but collagen type IV positive. Finally, within this model both MT1-MMP and MMP-2 appear to be involved in mediating the angiogenic response although their activity appears to be outside of the formation of a functional complex with αᵥβ₃.

What is claimed is:

1. A method for screening agents which inhibit an angiogenic response comprising
a) contacting:
i) an inactive pro form or convertase-activated form of an integrin ox subunit,
ii) an agent to be tested for the ability to inhibit angiogenesis, and
iii) metalloprotease MT1-MMP,
under conditions promoting an increase in activation of the integrin α subunit in the absence of said agent, and
b) correlating inhibition of said increase in integrin ox subunit activation with the ability of the agent to inhibit angiogenesis.

2. The method of claim 1 wherein the correlating step is accomplished by observing a difference in migration of the activated form versus the inactive form of the alpha subunit in electrophoresis or chromatography.

3. The method of claim 1 or 2 wherein the MT1-MMP and pro form of the integrin α subunit are recombinantly expressed within the same cell.

4. The method of claim 1 in which said contacting step is performed within a cell.

5. The method of claim 1 in which the activation of said alpha subunit is accomplished by cleavage of the pro form of said alpha subunit.

6. The method of any of the foregoing claims wherein the activation of said alpha subunit is accomplished by a change in glycolsylation of the pro form of said alpha subunit.

7. The method of claim 1 in which said correlating step comprises the use of a reporter gene and detection of the presence or absence of the product of reporter gene expression as an indication of inhibition of an increase in alpha subunit activation.

8. A method of treating a patient suffering from a pathological condition in which angiogenesis is at least partially a causative or perpetuating factor comprising administering to said patient an agent capable of inhibiting an increase in activation of an inactive pro form or convertase-activated form of an integrin ox subunit by MT1-MMP metalloprotease.

9. A method of treating a patient suffering from a pathological condition in which angiogenesis is at least partially a causative or perpetuating factor comprising treating said patient with agent that specifically inhibits activation of a pro form of a specific integrin α subunit selected from the group consisting of α₃, α₄, α₅, α₆, α₇, α₈, α₉, α_{2b}, α_{E} and α_{V}.

10. The method of claim 9 in which said specific integrin α subunit is α_{V}.
